(19) United States
(12) Patent Application Publication
(10) Pub. No.: US 2008/0262340 A1
(43) Pub. Date: Oct. 23, 2008

(54) Moving Table Mri

(75) Inventors: Cornelis Leonardus Gerardus Ham, Eindhoven (NL); Miha Fuderer, Eindhoven (NL); Johan Samuel Van Den Brink, Eindhoven (NL)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N.V., Eindhoven (NL)

(21) Appl. No.: 11/569,069

(22) PCT Filed: May 12, 2005
(86) PCT No.: PCT/IB2005/051559

§ 371 Date: Nov. 14, 2006

(30) Foreign Application Priority Data
May 14, 2004 (EP) 04102123.9

(51) Int. Cl. A61B 5/055; G01R 33/563

(52) U.S. Cl. 600/415

(57) ABSTRACT

The invention relates to a MRI system and to a method for producing an image with such an system. In order to provide a MR imaging technique with a high efficient MR signal acquisition, which provides a high level of comfort to a patient, a MRI system and method are suggested, where image data from an object are acquired while the object is moving with variable speed relative to the MRI system, and where the image data are combined and an image of the object is reconstructed.

The invention relates to magnetic resonance imaging (MRI) system. Furthermore the invention relates to a method for producing an image using a MRI system and to a computer program for producing such an image.

More particularly the invention relates to a technique to acquire magnetic resonance (MR) images in case of a large field of view (FOV). Especially if image data of a whole human body shall be acquired, the FOVs extend beyond the homogeneity of the magnet, beyond the gradient's linearity volume and beyond the transmit coil's sensitivity volume. It is known to produce such images from MR signals that are acquired using a “moving bed approach”, a technique which is particularly useful for contrast-enhanced MR angiography.

Two different methods of carrying out the “moving bed approach” are known: During the first method, known as “MOBITRAK”, a patient is moved between a number of stations within the MRI system and imaging is performed while the patient is situated in such a stationary position. A major disadvantage of this method is its time-inefficiency, i.e. a significant portion of time is taken by carrier movements, while no data is acquired. Furthermore the patient's comfort is reduced because of high acceleration and deceleration rates of the patient table, especially required when capturing the arterial phase in contrast-enhanced MR angiography studies. During the second method, known as “Continuously Moving Bed Imaging” or “COMBI”, imaging is continuously performed while the patient moves with a constant, relatively low, speed through the MRI system. The main advantage of this method is its higher level of patient's comfort. A major disadvantage of COMBI is its limited resolution and the high number of artefacts. All k-space lines required to capture a certain image resolution must be covered while the patient moves the distance of one system homogeneity volume (typically <30 cm). A typical allowed scan time for 120 cm (corresponding to peripheral vascular MR angiography coverage) being <50 seconds, only 10 seconds per 30 cm travel distance are allowed. This implies, with a profile acquisition time TR of 5 ms, that only 2000 k-values in Y- and Z-direction can be sampled. These lines are too few for adequate high-resolution MR imaging.

It is an object of the invention to provide a MR imaging technique with a high quality MR signal acquisition, which provides a high level of comfort to a patient.

This object is achieved according to the invention by a MRI system, the system comprising an object carrier movable within the MRI system, a control unit for controlling the MRI system, the control unit being adapted to control the movement of the object carrier, to derive the position of the object carrier, to acquire image data from an object, while the object is moving with variable speed relative to the MRI system. The MRI system further comprises a processing unit being adapted to reconstruct k-space MR data to images of the object parts, to combine the image data of the parts and to reconstruct an image of the full object.

The term “the object is moved with variable speed relative to the MRI system” does include a movement with a speed >0, as well as a “virtual” movement with a speed =0. In other words, the above term includes a case where the object is moving relative to the MRI system as well as a case where the object is stationary relative to the MRI system.

The MRI system include inter alia coils for creation of gradient magnetic fields, current supply devices, high frequency generators, control devices, RF signal antennae, readout devices etc. All appliances are adapted to carry out the method according to the present invention. All devices, e. g. the control unit and the processing unit, are constructed and programmed in a way that the procedures for obtaining data and for data processing run in accordance with the method of the invention.

The object of the present invention is also achieved by a method for producing an image with an MRI system, the method comprising the steps of positioning an object in the MRI system, controlling the movement of the object carrier, acquiring image data from the object, while the object is moving with variable speed relative to the MRI system, and combining the image data acquired and reconstructing an image of the object.

The object of the present invention is also achieved by a computer program comprising computer instructions adapted to perform the method according to the invention when the computer program is executed in a computer. The technical effects necessary according to the invention can thus be realized on the basis of the instructions of the computer program in accordance with the invention. Such a computer program can be stored on a carrier such as a CD-ROM or it can be available over the internet or another computer network. Prior to executing the computer program is loaded into the computer by reading the computer program from the carrier, for example by means of a CD-ROM player, or from the internet, and storing it in the memory of the computer. The computer includes inter alia a central processor unit (CPU), a bus system, memory means, e. g. RAM or ROM, storage means, e. g. floppy disk or hard disk units and input/output units. Preferably the computer is an integral component of the MRI system.

The present invention enables a high quality MR signal acquisition with a high level of patient comfort. This is achieved by combining stationary and moving acquisition of image data into a single procedure. That means that image data are acquired at variable object speed. In other words a MRI system and method are suggested, where image data from an object are acquired while the object is stationary as well as while the object is moving, and where the image data are combined and an image of the object is reconstructed.

The invention enables a high level of comfort to the patient, which is achieved by continuous, relatively slow, movement of the object and concurrent image data acquisition between any stationary positions of the object carrier (where data is being acquired). If the time during stationary positions of the object is used for acquiring image data the need to apply high acceleration and deceleration rates is reduced.

These and other aspects of the invention will be further elaborated on the basis of the following embodiments which are defined in the dependent claims.

In a preferred embodiment of the invention the control unit is adapted to drive the object carrier according to the part of k-space being acquired. In another preferred embodiment the control unit is adapted to acquire image data from an object, while the object is stationary relative to the MRI system, and to acquire image data from the object, while the object is moving relative to the MRI system.

In yet another preferred embodiment of the invention the control unit is adapted to acquire image data depending on the position of the object. Preferably image data is acquired during periods in which the object is moving at low velocity near a stationary position. This technique is an enhancement to MOBITRAK, an enhancement that uses the fact that the magnetic field homogeneity (and gradient linearity and coil sensitivities) do not drop sharply at the edge of some “homogeneity volume”, but that the regions slightly outside of a high-homogeneity region can also be used for imaging. Since the data from these regions sees slightly higher distortions, preferably only high k-values are acquired during periods of displacement. On the other hand, no image data acquisition is performed during periods of significant displacement of the object relative to a station. In this embodiment of the invention the position of the field of view is adapted according to the position of the object and/or the velocity of the object by either shifting in hybrid X-k space or adjusting the RF frequency offset to compensate for offcenter variations of the moving carrier.

In another preferred embodiment of the invention, the field of view is stationary with respect to the magnet. The control unit is adapted to apply higher object-velocities depending on the k-value region that is being acquired. This is an enhancement to the COMBI technique. As a result, the efficiency of the MR image acquisition can be enhanced. During periods of slow movement, the number of motion artefacts is reduced, if low k-values are acquired during periods of low velocity, while high k-values are acquired during periods of high velocity of the object. In a specific case, the carrier is in stationary position for the central 80% of k-space, while the high k-values corresponding to 20% of k-space coverage are acquired while the carrier is moving. In yet another case, where k-space is covered a-symmetrically (applying partial fourier) the object carrier starts moving when the k-value(s) exceed a pre-defined threshold. The threshold is derived by the ratio of the allowed scan time per station, plus the time required for travelling to the next station, and the fraction of the time required for movement that can be used for data acquisition. In a typical example, the travel time amounts up to 6 seconds, 50% whereof can be used for data sampling (25% on both sides of the station), and the total allowed time for that station is given as 15 seconds. This implies that data acquisition takes place under stationary conditions for 9 seconds, and (0.5*6=) 3 seconds under movement. Assuming uniform circular k-space coverage (along Y and Z direction), it is understood that the carrier moves for k-space lines where |(kY,kZ)|>(1−3/(9+3))*|(kY,kZ)|MAX=0.75*|(kY,kZ)|MAX. Similar rules can be derived for different k-space coverage strategies.

According to a further embodiment of the invention the focus is directed to resolution requirements of a MR scan. Naturally, some parts of the object to be scanned require a higher resolution than other parts of the object. In case the object to be scanned is a human body, the outer, peripheral parts of the body, i.e. head-neck region and feet/calf region, usually require a higher resolution scanning than the central part of the body (due to inherently smaller structures of interest, e. g. vasculature). It has been found, that imperfections of MR systems (e. g. broadening of the point spread function, ghosting) compromise the resolution of the COMBI technique. So, for example, acquiring very high k-value lines using the COMBI approach does not significantly improve the true resolution of the images. According to the present invention a combination of stationary scans and scans during movement of the object solves this problem. In other words, different parts of the object are acquired with acquisition methods that reflect the needs for different coverage and resolution for the different object parts.

In this embodiment of the invention the control unit is adapted such, that the peripheral parts are scanned during a period in which the object is in a stationary position, thereby acquiring high resolution image data. This prevents motion blurring that would occur during the COMBI method. The acquired image data comprises a fall k-space, i.e. high and low k-space lines. Furthermore the control unit is adapted such, that image data for the central part of the object are acquired using e. g. the known COMBI method or a comparable method with variable, non-zero, speed, taking into account the fact, that resolution requirements with regard to the central part of the object are lower than for its outer parts. The acquired image data again comprises a full k-space, where the highest values of k are less than for acquisitions at stationary positions. Using the above embodiment a relatively quick movement is possible, e. g. to follow a contrast bolus within the object, which is especially useful e. g. for contrast enhanced MR angiography.

These and other aspects of the invention will be described in detail hereinafter, by way of example, with reference to the following embodiments and the accompanying drawings; in which:

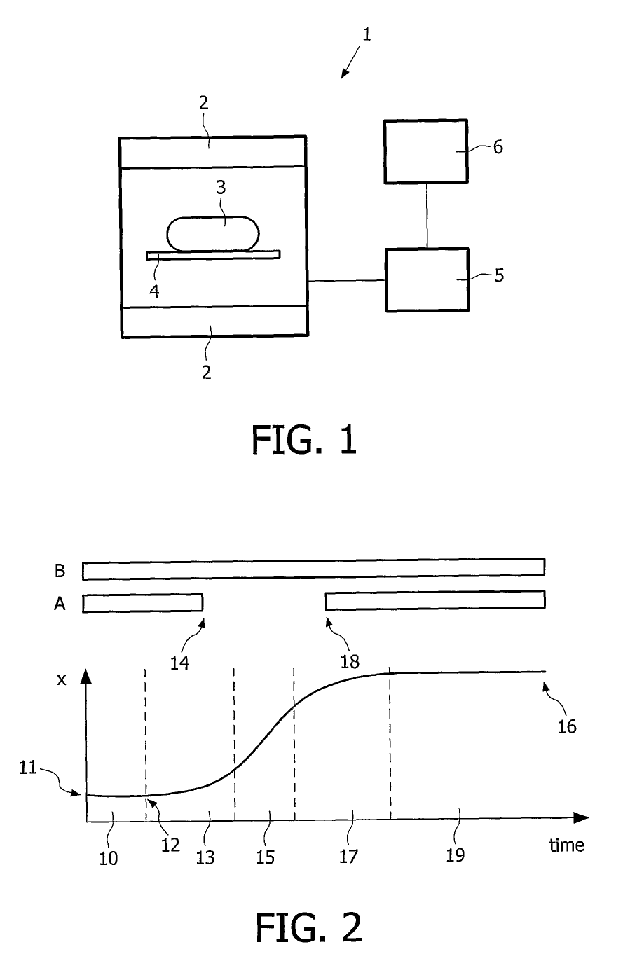

FIG. 1 is a block diagram showing the MRI system according to the invention;

FIG. 2 is a graphic representation of the image data acquisition according to the invention.

A MRI system on which the preferred embodiment can be implemented is shown in a simplified block diagram of FIG. 1. The MRI system 1 comprises inter alia coils 2 for creation of gradient magnetic fields, RF signal antennae, readout devices, current supply devices, high frequency generators etc. An object 3 is placed within the magnet on an object carrier 4. The MRI system 1 further comprises a control unit 5 and a processing unit 6. The control unit 5 is adapted for providing the MRI system 1 with specific scan parameters. It includes a computer console with input and output devices, e. g. a computer monitor and a keyboard. Other input devices, e. g. touch screen or mouse might be used as well. The control unit 5 is adapted to control the movement, and determine the position, of the object carrier 4 and to control the image data acquisition. Movement control preferably includes means to vary and to control the motion velocity and to drive the carrier according to the part of k-space being acquired. Furthermore control unit 5 is adapted to select k-space parts according to the position of the carrier. For this purposes the control unit 5 includes a computer including CPU, memory and storage means etc. The computer comprises a computer program adapted to perform the inventive method. The processing unit 6 is adapted for combining the image data acquired and to reconstruct an image of the object 3. The processing unit 6 includes a computer comprising a computer program adapted to perform these steps.

Preferably the processing unit 6 is adapted to reconstruct k-space MR data to images of the object parts, to combine the image data of the parts and to reconstruct an image of the full object. In another embodiment of the invention the images of the object parts are reconstructed and displayed on the monitor while moving without reconstructing an image of the full object.

FIG. 2 shows an object carrier position vs. time curve, which is divided into five intervals. The first interval 10 corresponds to a period of image data acquisition, where the object carrier 4 is in its first stationary position 11 (start position). After the start 12 of the movement of the object carrier 4 the second interval 13 starts with a low acceleration of the object carrier 4. During this first part of the acceleration the object 3 is still relatively close to its original static stationary position 11, e. g. less than 5 cm.

According to this embodiment the MRI system is adapted to acquire image data in a method similar to MOBITRAK. However, only part of the acquisition of every station is done under stationary position. By preference, lower k-values are acquired during stationarity. Another part of the data of a “station” is done during motion. Thereby the position of the FOV is adapted depending on the position of the object 3 during periods of low acceleration of the object 3. For this purpose the FOV is moved with the object, either by shifting the MR data in the processing module to align the data in hybrid X-k space, or by adapting the RF frequency offset of the transmit and receive paths. In other words the MRI system 1 adapts its transmit frequency, demodulation frequency, etc. in order to compensate the object's movement. This is performed, until a large part of the FOV is in the inhomogeneous part of the magnet, non-linear part of the gradient coil and/or insensitive part of the RF transmit/receive coil. For large displacement with regard to the original static stationary position 11 degradation of the image quality might occur. Therefore the scanning at large displacement is done with high k-values, that generally contain less MR signal.

In this embodiment, the data acquisition stops at a certain point in time 14, when the object carrier 4 reaches a certain velocity. Subsequent to the second interval 13 a third interval 15 follows, corresponding to a constant speed of the object carrier 4. If the object carrier 4 approaches its second stationary position 16, the deceleration interval 17 starts. At a certain point in time 18, when the object carrier 4 again reaches a certain velocity, the data acquisition starts again. For this deceleration the same procedure is applied as for the acceleration part of the movement. Finally in the fifth interval 19 the object carrier 4 is in its second stationary position 16, where the data acquisition continues. The scan time corresponding to this embodiment of the invention is illustrated using the bar “A” above the speed curve.

If the gradient and RF units of the MRI system 1 are left in a running mode, a steady state of the eddy currents and/or the spin system can be maintained. Another advantage is that the patients comfort is improved since acoustic noise continues to be present.

According to another embodiment of the invention the FOV remains at the same position relative to the magnet during data acquisition while the object is (or may be) moving. Comparable to COMBI, an (x, ky,kz) space is filled with data, where x is parallel to the direction of motion. Again, the scan time here is illustrated by the bar “A”. In this embodiment of the invention image data acquisition may be performed during the entire movement. The scan time corresponding to this embodiment of the invention is illustrated using the bar “B” above the speed curve. Thereby the movement with high velocity of the object carrier corresponds with a high probability of induced motion artefacts. Therefore high k-values are acquired during periods with highest velocity, whereas low k-values are acquired at low speed. In a special case of this embodiment, no acquisition is done during high velocity.

In another embodiment, the whole body of a human object is scanned for e. g. total body angiography. Thereby the image data acquisition of the head-neck region is performed with a stationary object carrier. From then on, the object carrier moves continuously thereby allowing only relatively short scan times. For the feet/calf region, where high-resolution is required again, image data acquisition is performed using a stationary object carrier. Thereby the outer stations are covered in a single stationary acquisition to achieve long scan times for high resolution. Typically, the outer stations have a dedicated receive coil to enhance SNR and to allow for parallel imaging (SENSE) at high reduction factors. The central part of the object is preferably not covered by dedicated surface coils for signal reception. Here, the quadrature body coil, or a large-volume phased array coil (embedded with the system) is used for signal reception.

In another embodiment peripheral vascular angiography is performed. Thereby the upper station can either correspond to the pelvic station, or may provide a “BolusTrak” (contrast enhanced timing) acquisition in the thorax area. As contrast arrival time is hard to predict, pre-defining the allowed displacement under COMBI is difficult for a contrast arrival detection scan. Here, a stationary carrier position is preferred, which is easily achieved by the inventive method.

A typical procedure description for peripheral vascular angiography, including BolusTrak, using the inventive approach is as follows: First a mask image of the lower station (feet-calf) is acquired, typically by a 1-2 minutes scan with elliptical-centric k-space ordering as described in U.S. Pat. No. 6,577,127. In a next step the object carrier movement is started. Then, under continuous movement, the mask FOV up to the aortic bifurcation is acquired, with typically 10 seconds acquisition time per FOV corresponding to the system's homogeneity volume (typically <30 cm). If the object carrier has reached the second stationary position the object carrier is stopped and the final station's mask is acquired with a stationary object carrier. Afterwards a scan is performed to detect the arrival of the contrast bolus, e. g. by employing a time-resolved scan with in-line subtraction of background data (BolusTrak). Upon detecting arrival of contrast, the contrast-enhanced upper station is acquired using stationary object carrier. In a next step the object carrier movement is started again. Under continuous movement, the contrast enhanced FOV is acquired down to the calf-feet station, where the object carrier is stopped again and the contrast enhanced lower station is acquired using the 1-2 minutes scan. The sequence becomes especially efficient when a relatively large number of stations would be required due to small system homogeneity volumes.

In the following the known MOBITRAK approach will be compared with the method according to the invention. Carrier velocity and acceleration rates are taken from state-of-the-art equipment to be 180 mm/s and 150 mm/s2, respectively.

In the first example a 300 mm FOV, 1200 mm coverage and four stations are employed, where BolusTrak is used at the upper station position. With the known MOBITRAK approach the time requirements are as follows: upper station 15 seconds; two middle stations 10 seconds; lower station 90 seconds; three inter-station latencies: 300 (mm)/180 (mm/s)+180 (mm/s)/150 (mm/s2)=2.9 sec; total time 134 seconds. With the method according to the present invention the time requirements are as follows: upper station 15 seconds; time to acquire two middle stations: 3*10=30 seconds; lower station 90 seconds; total time 135 seconds.

In a second example a 300 mm FOV, 1800 mm coverage and six stations are employed. With the known MOBITRAK approach the time requirements are as follows: upper station 25 seconds; four middle stations 11 seconds; lower station 90 seconds; five inter-station latencies: 300 (mm)/180 (mm/s)+180 (mm/s)/150 (mm/s2)=2.9 sec; total time 174 seconds. With the method according to the present invention the time requirements are as follows: upper station 25 seconds; time to acquire four middle stations: 5*11=55 seconds; lower station 90 seconds; total time 170 seconds.

In a third example a 300 mm FOV, 1200 mm coverage and four stations are employed, where BolusTrak is used at the upper station position. With the known MOBITRAK approach the time requirements are as follows: upper station 10 seconds; two middle stations 10 seconds; lower station 90 seconds; three inter-station latencies: 300 (mm)/180 (mm/s)+180 (mm/s)/150 (mm/s2)=2.9 sec; total time 129 seconds. Thereby the object carrier moves to the first station during BolusTrak switching time and breath hold instruction. With the method according to the present invention the time requirements are as follows: time to acquire three stations: 4*10=40 seconds; lower station 90 seconds; total time 130 seconds.

It is appreciated that the combined stationary and moving method is very time efficient. Only for a three station acquisition (system homogeneity volume of approximately 40 cm), MOBITRAK is more time efficient. Nevertheless, the 5-10 seconds longer scan time if the method according to the present invention is used are well acceptable to reduce the patient discomfort related to high acceleration/deceleration rates.

A significant advantage of the inventive method is that it does not require additional table stroke. Further advantages are that it is easy to implement a sensitivity encoding (SENSE) scan, where the spatial information related to the coils of a receiver array are utilized for reducing conventional Fourier encoding, since the central part of the object (imaged under continuous motion) does not require SENSE to achieve adequate resolution and coverage in 10-15 seconds scan time. Thus, SENSE is only applied in outer stations, which require a short breath-hold, or a very high resolution.

It will be evident to those skilled in the art that the invention is not limited to the details of the foregoing illustrative embodiments, and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein. It will furthermore be evident that the word “comprising” does not exclude other elements or steps, that the words “a” or “an” does not exclude a plurality, and that a single element, such as a computer system or another unit may fulfil the functions of several means recited in the claims. Any reference signs in the claims shall not be construed as limiting the claim concerned.

1. A magnetic resonance imaging (MRI) system, the system comprising
an object carrier movable within the MRI system,
a control unit for controlling the MRI system, the control unit being adapted
to control the movement of the object carrier,
to derive the position of the object carrier,
to acquire image data from the object, while the object is moving with variable speed relative to the MRI system, and
a processing unit being adapted
to combine the image data acquired and
to reconstruct an image of the object.

2. The MRI system as claimed in claim 1, wherein the control unit is adapted to drive the object carrier according to the part of k-space being acquired.

3. The MRI system as claimed in claim 1, wherein the control unit is adapted
to acquire image data from an object, while the object is stationary relative to the MRI system, and
to acquire image data from the object, while the object is moving relative to the MRI system.

4. The MRI system as claimed in claim 1, wherein the control unit is adapted to acquire image data of a well-defined portion of the object, whereby part of that data is acquired during displacement of that portion of the object with respect to a default position or a stationary position.

5. The MRI system as claimed in claim 1, wherein the control unit is adapted to acquire image data while the object shows a small displacement from the default position or the stationary position.

6. The MRI system as claimed in claim 1, wherein the control unit is adapted to shift the field of view in accordance to the displacement.

7. The MRI system as claimed in claim 1, wherein the control unit is adapted to acquire low k-values for the stationary part and high k-values for displacement.

8. MRI system as claimed in claim 1, wherein the control unit is adapted to select k-space parts according to the position of the object carrier.

9. The MRI system as claimed in claim 1, wherein the control unit is adapted
to keep the field of view fixed with respect to a magnet of the MRI system and
to perform frequency-encoding parallel to the motion direction, and wherein the processing unit is adapted
to perform a fourier-transformation in motion-direction of each profile, and
to fill the data in (x,ky,kz)-space.

10. The MRI system as claimed in claim 1, wherein the control unit is adapted to acquire high k-values during movement with high velocity and to acquire low k-values during movement with low velocity.

11. The MRI system as claimed in claim 1, wherein the control unit is adapted to acquire high-resolution image data of part of the object while the object is in a stationary position and to acquire image data of other part of the object with lower resolution while the object is moving.

12. The MRI system as claimed in claim 1, wherein the control unit is adapted to acquire very high k-space values while the object is in a stationary position.

13. The MRI system as claimed in claim 1, wherein multi-element RF receive coils are used for the object parts corresponding to the stationary positions.

14. The MRI system as claimed in claim 1, wherein multi-element RF receive coils are used for any object part.

15. A method for producing an image with an MRI system, the method comprising the steps of:
positioning an object in the MRI system,
controlling the movement of the object carrier,
acquiring image data from the object, while the object is moving with variable speed relative to the MRI system, and
combining the image data acquired and reconstructing an image of the object.

16. A computer program for producing an image of an object using a MRI system, the computer program comprising
computer instructions for controlling the movement of the object carrier,
computer instructions for acquiring image data from the object, while the object is moving with variable speed relative to the MRI system, and
computer instructions for combining the image data acquired and for reconstructing an image of the object.
